(19) United States
(12) Patent Application Publication
(10) Pub. No.: US 2011/0251432 A1
(43) Pub. Date: Oct. 13, 2011

(54) SYNTHESIS OF [18F]-LABELLED ALKYL MESYLATES USING FLUOROUS SPE SEPARATION

(76) Inventors: Bengt Langstrom, Uppsala (SE); Tor Kihlberg, Uppsala (SE)

(21) Appl. No.: 12/673,635

(22) PCT Filed: Aug. 27, 2008
(86) PCT No.: PCT/US08/74363

§ 371 Date: Jun. 28, 2011

Related U.S. Application Data
(60) Provisional application No. 60/968,243, filed on Aug. 27, 2007.

(51) Int. Cl. C07C 209/22; C07C 319/00; C07C 17/00; C07C 41/16

(52) U.S. Cl. 564/468; 568/683; 568/56; 570/140

(57) ABSTRACT

The invention relates to new processes for preparation of ¹⁸F-labelled alkylation reagents that can be used in the alkylation of amines that are suitable for use in labelling of Positron Emission Tomography (PET) radiotracers. (I)

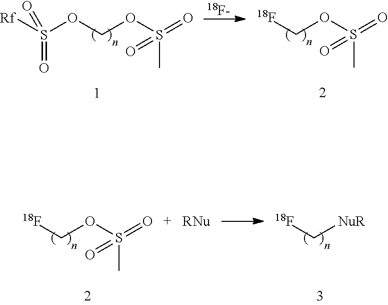

### FIELD OF THE INVENTION

The invention relates to new processes for preparation of [¹⁸F]-labelled alkyl mesylates using fluorous solid phase extraction (SPE) suitable for use in labelling of Positron Emission Tomography (PET) radiotracers.

### BACKGROUND

The favored radioisotope for PET, ¹⁸F, has a relatively short half-life of 110 minutes. ¹⁸F-labelled tracers for PET therefore have to be synthesised and purified as rapidly as possibly, and ideally within one hour of clinical use. PET tracers are frequently labelled with [¹⁸F]fluoroalkyl groups to produce [¹⁸F]fluoroalkylated PET tracers. [¹⁸F]fluoroalkyl mesylates are important reagents for performing O-, N-, and S-[¹⁸F]fluoroalkylations, such as [¹⁸F]fluoromethylations, and are commonly used to radiolabel radiotracers for use in PET studies.

[¹⁸F]Fluoroalkyl mesylates have previously been prepared by nucleophilic displacement, by [¹⁸F]F⁻, of a leaving group from a suitable precursor compound. For example, in Comagic et al, Applied Radiation and Isotopes (2002), 56, 847-851 a 2 bromo-1-[¹⁸F]fluoroethane is prepared by nucleophilic displacement of 1,2 dibromoethane with ¹⁸F⁻. Solid-phase preparations of [¹⁸F]fluoroalkyl halides are described in WO 2004/056726 which discloses a process for preparation comprising the treatment of a solid support-bound precursor of the formula solid support linker-SO₂-O-(CH₂)ₙX, wherein n is an integer from 1 to 7 and X is chloro, bromo or iodo, with ¹⁸F⁻.

Unfortunately, production of [¹⁸F]fluoroalkylation reagents, such as the corresponding mesylate, is complicated. A few of the drawbacks with existing processes are complicated purification steps, relatively long preparation times and non-optimal yields.

Therefore. in view of the importance of [¹⁸F]fluoroalkyl mesylates as radiolabelling reagents, there exists a need for new synthetic methods for their preparation in high radiochemical yield and of high purity as well as a need of a robust, easily automated system that can be used in the alkylation of amines.

Discussion or citation of a reference herein shall not be construed as an admission that such reference is prior art to the present invention.

### SUMMARY OF THE INVENTION

There is a need of a robust, easily automated and high yielding synthesis of 18F-labelled alkylation reagents that can be used in the alkylation of amines.

One aspect of the present invention sets forth the yielding synthesis of 18F-labelled alkylation reagents from fluorous tagged bis sulphonic esters and thereafter purified using fluorous-SPE (solid phase extraction) separation.

In a first aspect, the present invention provides a process for the preparation of structure 3

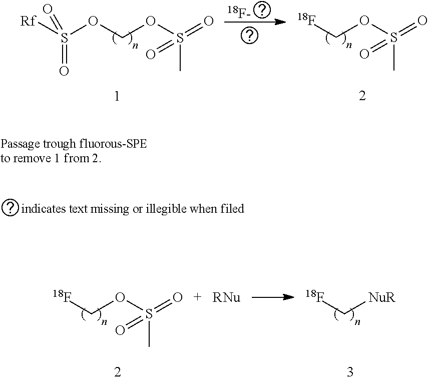

comprising the steps of

i) treating structure 1 with ¹⁸F-fluoride to generate structure 2,

ii) optionally using kypotofix or an ionic liquid to speed the reaction from structure 1 to structure 2; then

iii) passing structure 1 through a fluorous-SPE to obtain structure 2, and finally

iv) obtaining structure 3 through a nucleophilic reaction wherein n is equal to or greater than 1.

Yet another embodiment of the present invention demonstrates that the nucleophile is Nu and Nu is either NH₂, HNR′, O—, S—, or a stabilized carbanion. Additionally, an embodiment of the invention shows that n is at the least 1.

A further embodiment of the present invention shows that the ionic liquid is ethylammonium nitrate or sodium chloride. A further embodiment of the invention depicts that when using kypotofix or an ionic liquid to speed the reaction from structure 1 to structure 2 that rate of speed increases the reaction from obtaining structure 1 from structure 2 by at least 60% with less than a 2% loss of radiochemical purity of structure 2.

Still a further embodiment of the invention shows that the nucleophile may be electrically neutral or negatively charged.

Another embodiment of the present invention depicts a radiopharmaceutical kit for the preparation of structure 3 for use in fluorous PET chemistry, which comprises

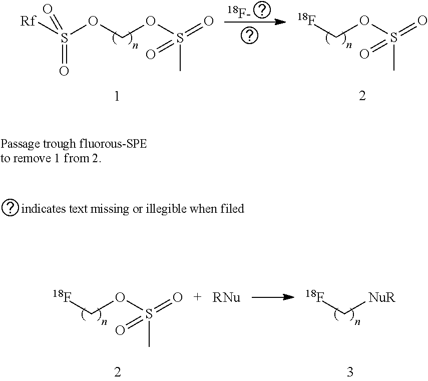

i) treating structure 1 with ¹⁸F-fluoride to generate structure 2,

ii) optionally using kypotofix or an ionic liquid to speed the reaction from structure 1 to structure 2; then

iii) passing structure 1 through a fluorous-SPE to obtain structure 2, and finally

iv) obtaining structure 3 through a nucleophilic reaction wherein n is equal to or greater than 1.

Still another embodiment of the present invention shows a method for the use of preparing structure 3.

### DETAILED DESCRIPTION OF THE INVENTION

Compound 1 is an alkyl chain having a mesylate in one end and a perfluorinated alkyl sulfonate ester in the other end. The perfluorinated alkyl sulfonate ester should have similar reactivity as a triflate group (trifluoromethyl sulfonate ester). Curran, D. P. Fluorous reverse phase silica gel. A new tool for preparative separations in synthetic organic and organofluorine chemistry, Synlett, 2001, pgs. 1488-1496.

Thus, in a nucleophilic substitutions reaction the reaction rate of the perfluorinated alkyl sulfonate ester moiety should be at least two orders of magnitude higher than that of the mesylate moiety. Therefore, [¹⁸F]F⁻ will predominantly substitute the perfluorinated alkyl sulfonate ester. By passage through a column containing a perfluorinated alkyl matrix, structure 1 will be retained and separated from structure 2.

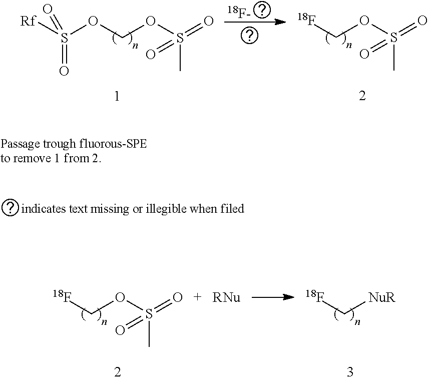

There are several advantages with the present method and system. The highly reactive perfluorinated alkyl sulfonate ester should give rapid and efficient incorporation of [¹⁸F]F⁻ using small amounts of structure 1. Yet another advantage is that the well known fluorous-SPE purification should be easy to automate and should give an efficient separation of structure 1 from structure 2.

Still other advantages are achieved in that the low concentration of structure 1 in the fluorous-SPE purified structure 2 should allow for use of small amounts of the precursor nucleophile (RNu). Another advantage of the present invention is the high reactivity of the mesylate, structure 2 (approximately 1000 times higher than the corresponding iodide) should allow for use of small amounts of the precursor nucleophile (RNu) and rapid labeling.

The embodiment of obtaining structure 2 from structure 1 is preferably carried out without any solvents, but addition of any solvents that would promote the reaction could be included. Suitable solvents would be e.g. acetonitrile, dichloromethane (DCM), dimethylformamide (DMF), dimethyl sulfoxide (DMSO) and tetrahydrofurane (THF). Structure 1 is passed through a fluorous solid-phase extraction column containing a perfluorinated alkyl matrix, wherein structure 1 will be retained and separated from structure 2.

A further embodiment of the present invention depicts that the addition of [¹⁸F]F⁻ will predominantly substitute the perfluorinated alkyl sulfonate ester. Krytofix 2.2.2 (also known as 4,7,13,16,21,24 hexaoxa-1,1 0-diazabicyclo[8,8,8] hexacosane) and an ionic liquid help speed the reaction rate up to at least 60% faster as compared to not using them to separate structure 1 to form structure 2. An ionic liquid is used herein for salts whose melting point is relatively low (below 100° C.). Examples of ionic liquids would be ethylammonium nitrate or sodium chloride.

Another embodiment of the present invention shows a nucleophilic substitution reaction of structure 2 wherein the reaction rate of the perfluorinated alkyl sulfonate ester moiety should be at least two orders of magnitude higher than that of the mesylate moiety. Therefore, [¹⁸F]F⁻ will predominantly substitute the perfluorinated alkyl sulfonate ester. A nucleophilic substitution reaction is defined herein as a fundamental class of substitution reactions in which an “electron rich” nucleophile selectively bonds with or attacks the positive charge of a group or atom called the leaving group. The nucleophile may be electrically neutral or negatively charged, whereas the substrate is typically neutral or positively charged.

As mentioned earlier, structure 1 is an alkyl chain having a mesylate on one end and a perfluorinated alkyl sulfonate ester on the other end. As shown below, structure 1 reacts via a nucleophilic substitution reaction in a fluorous-SPE (Solid-Phase Extraction) column, structure 2 will be formed. Fluorous Solid Phase Extraction (F-SPE) is used to quickly separate fluorous compounds from non-fluorous compounds in three easy steps. First, the reaction mixture is loaded onto the column. Second, the non-fluorous compounds are eluted with a fluorophobic solvent in one fraction. Third, the fluorous compounds are eluted with a fluorophilic solvent.

Additionally, the substitution reaction of structure 1 reacts with 18F to substitute the perfluorinated alkyl sulfonate ester moiety with 18F. The optional use of kryptofix 2.2.2 and an ionic liquid aid in the rapid incorporation of 18F in place of the perfluorinated alkyl sulfonate ester (since rapid incorporation occurs at least two magnitudes higher than on the ester side chain than that of the mesylate moiety) into structure 1. After structure 2 is formed it undergoes a further reaction with a precursor nucleophile (RNu) to form structure 3 as shown below.

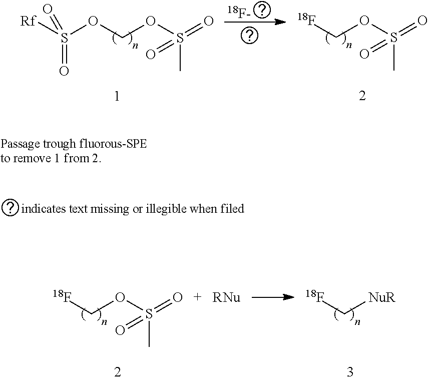

The high reactivity of the mesylate moiety in structure 2 is 1000 times high than the corresponding iodide in [11C] methyl iodide. This high reactivity allows for use of small amounts of the precursor nucleophile (RNu) and rapid labelling.

In order to further increase the reactivity of the fluoride, a phase transfer catalyst such as an aminopolyether or crown ether, for example, (Kryptofix 2.2.2.) is optionally added and the reaction performed in a non protic solvent. These conditions give reactive fluoride ions. Optionally, a free radical trap may be used to improve fluoridation yields, as described in WO 2005/061415. The term “free radical trap” is defined as any agent that interacts with free radicals and inactivates them. A suitable free radical trap for this purpose may be selected from 2,2,6,6-Tetramethylpiperidine-N-Oxide (TEMPO), 1,2-diphenylethylene (DPE), ascorbate, para-amino benzoic acid (PABA), a-tocopherol, hydroquinone, di-t-butyl phenol, f3-carotene and gentisic acid. Preferred free radical traps for use in the process of the invention are TEMPO and DPE, with TEMPO being most preferred.

The purity of structure 2 obtained from the F-SPE process is of at least 94% and most preferably at least 98%, without performing any additional purification of the product. The purity of structure 3 retains the purification of which is obtained from structure 2. Yet another embodiment of the present invention depicts a process as claimed in claim 1 where Rf is n-CxFy wherein x is 1-12 and y is 3-22.

One benefit of this process of preparation, from conventional methods, is that some of the starting reagent is converted into a suitable solvent and that the other reagents are non-volatile, making separation from any reagents and bi-products easy. The process of preparation hence provides a method of preparing [¹⁸F]fluoroalkyl halides of high purity in an uncomplicated process.

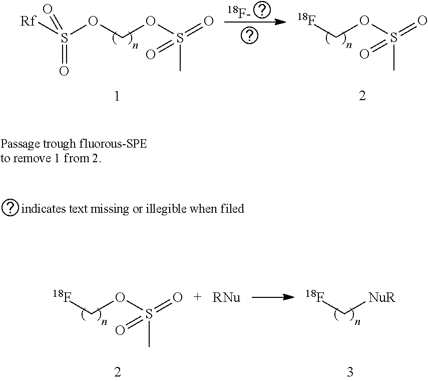

wherein the steps comprise of:

v) treating structure 1 with ¹⁸F-fluoride to generate structure 2,

vi) optionally using kypotofix 2.2.2 or an ionic liquid to speed the reaction from structure 1 to structure 2; then

vii) passing structure 1 through a fluorous-SPE to obtain structure 2, and finally

viii) obtaining structure 3 through a nucleophilic reaction wherein n is equal to or greater than 1.

Still a further embodiment of the present invention depicts both a method for the use of and the use of preparing structure 3 according to:

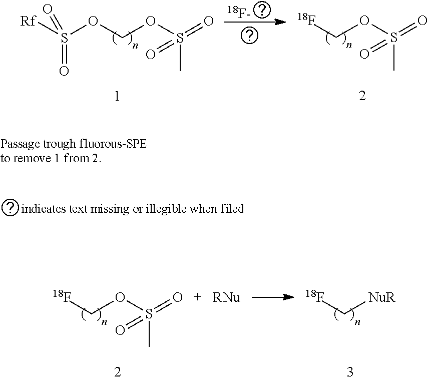

wherein the steps comprise of

i) treating structure 1 with ¹⁸F-fluoride to generate structure 2,

ii) optionally using kypotofix 2.2.2 or an ionic liquid to speed the reaction from structure 1 to structure 2; then

iii) passing structure 1 through a fluorous-SPE to obtain structure 2, and finally

iv) obtaining structure 3 through a nucleophilic reaction wherein n is equal to or greater than 1.

The invention is further described in the following examples, which is in no way intended to limit the scope of the invention.

### EXAMPLES

### Example 1

### Synthesis in Obtaining Structure 2

Optionally adding a solvent to structure 1 would speed up the reaction from structure 1 to structure 2 but some radiochemical purity of structure 2 may be lost. Suitable solvents would be e.g. acetonitrile, dichloromethane (DCM), dimethylformamide (DMF), dimethyl sulfoxide (DMSO) and tetrahydrofurane (THF). Structure 1 is passed through a fluorous solid-phase extraction column containing a perfluorinated alkyl matrix, wherein structure 1 will be retained and separated from structure 2. A further additive to structure 1 of the present invention depicts the addition of [¹⁸F]F⁻ which will predominantly substitute the perfluorinated alkyl sulfonate ester. Krytofix 2.2.2 (also known as 4,7,13,16,21,24 hexaoxa-1,1 0-diazabicyclo18,8,81 hexacosane) and an ionic liquid help speed the reaction rate up to two times as fast as prior reactions as well as separating structure 1 to form structure 2. An ionic liquid is used herein for salts whose melting point is relatively low (below 100° C.). Examples of ionic liquids would be ethylammonium nitrate or sodium chloride.

### Example 2

### Synthesis of Obtaining Structure 3 from Structure 2

Structure 2 undergoes a reaction with a precursor nucleophile (RNu) to form structure 3. The precursor nucleophile can either be NH2, HNR′, O—, S—, or a stabilized carbanion.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A process for the preparation of structure 3 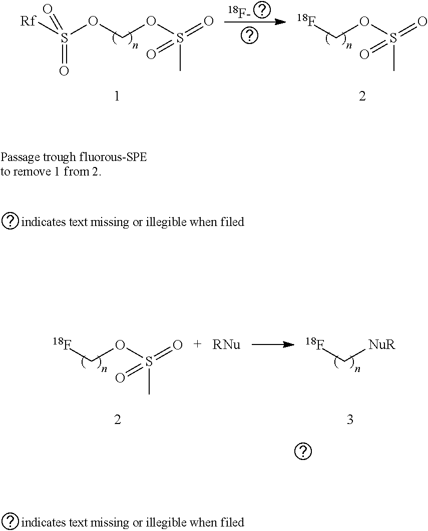
comprising the steps of
v) treating structure 1 with ¹⁸F-fluoride to generate structure 2,
vi) optionally using kypotofix 2.2.2 or an ionic liquid to speed the reaction from structure 1 to structure 2; then
vii) passing structure 1 through a fluorous-SPE to obtain structure 2, and finally
viii) obtaining structure 3 through a nucleophilic reaction wherein n is equal to or greater than 1.

2. A process as claimed in claim 1 wherein Nu is NH₂, HNR′, O—, S—, or a stabilized carbanion.

3. A process as claimed in claim 1 where Rf is n-CxFy wherein x is 1-12 and y is 3-22.

4. A process as claimed in claim 1 wherein the ionic liquid is ethylammonium nitrate or sodium chloride.

5. A process as claimed in claim 1 wherein the nucleophile is electrically neutral or negatively charged.

6. A process as claimed in claim 1 wherein solvents can be added to the reaction going from structure 1 to structure 2.

7. A process as claimed in claim 6 wherein the solvents are acetonitrile, dichloromethane (DCM), dimethylformamide (DMF), dimethyl sulfoxide (DMSO) and tetrahydrofurane (THF).

8. A radiopharmaceutical kit for the preparation of structure 3 for use in fluorous PET chemistry, according to claim 1.

9. A method for the use of preparing structure 3 according to claim 1.

10. The use of preparing structure 3 according to claim 1.
